(19) United States
(12) Patent Application Publication
(10) Pub. No.: US 2009/0137658 A1
(43) Pub. Date: May 28, 2009

(54) BENZENESULFONIC ACID SALT COMPOUNDS

(75) Inventors: Masataka HIKOTA, Shiki-shi (JP); Toru Iijima, Toda-shi (JP); Michael S. McClure, Cary, NC (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Tanabe Seiyaku Co.,Ltd.

(21) Appl. No.: 11/937,924

(22) Filed: Nov. 9, 2007

Related U.S. Application Data
(63) Continuation of application No. 10/837,579, filed on May 4, 2004, now abandoned.
(60) Provisional application No. 60/468,329, filed on May 6, 2003.

(51) Int. Cl. A61K 31/40; C07D 295/182

(52) U.S. Cl. 514/423; 548/540

(57) ABSTRACT

Besylate salts of trans-4-({2-[(2S)-2-cyano-pyrrolidinyl]-2-oxoethyl}amino)-N,N-dimethylcyclohexanecarboxamide are described as well as methods of using the same in the treatment of disorders characterized by hyperglycemia.

This is a continuation of U.S. application Ser. No. 10/837,579, filed May 4, 2004, now abandoned, which claims the benefit of U.S. provisional application No. 60/468,329, filed May 6, 2008, the contents of all of which are incorporated herein by reference.

### BACKGROUND OF THE INVENTION

1. Field of the Invention

The present invention relates to anhydrate and monohydrate benzenesulfonic acid salts. In particular, to the anhydrate and monohydrate forms of the benzenesulfonic acid salt of (2S)-2-cyanopyrrolidinyl-2-oxoethyl amine derivatives. These compounds are inhibitors of serine proteases, such as dipeptidyl peptidases, and are useful in the treatment of disorders such as hyperglycemia and/or other conditions of diabetes. The particular forms disclosed herein demonstrate unexpectedly beneficial physical properties for use as commercial medicaments.

2. Description of the Related Art

International Patent Application PCT/JP01/08803 filed May 10, 2001, and published as WO02/30891 on 18/Apr./2002, discusses inhibitors of serine proteases including Dipeptidyl Peptidase IV (DPP IV), and discloses aliphatic nitrogen-containing 5-membered ring compounds which demonstrate such activity, including trans-4-({2-[(2S)-2-cyanopyrrolidinyl]-2-oxoethyl}amino)-N,N-dim ethylcyclohexanecarboxamide. However, several salts of these compounds have been found to absorb very large amounts of water at the expected exposure humidities if utilized in a medicament (e.g., 20-75% relative humidity (RH)). As a result, suitability of the compound as a medicament could be compromised unless there were special handling and storage procedures instituted.

### SUMMARY OF THE INVENTION

The present inventors have now identified novel benzenesulfonic acid salts of (2S)-2-cyanopyrrolidinyl-2-oxoethyl amines, which are suitable as serine protease inhibitors. These benzenesulfonic acid salts have moisture sorption properties superior to the HCl salts of (2S)-2-cyanopyrrolidinyl-2-oxoethyl amines disclosed in the art. The compounds may be prepared in crystal form and therefore have good physical stability. That is, the benzenesulfonic acid salts of the present invention sorb much lower amounts of water when exposed to a broad range of humidities and can be prepared in a physically stable crystal form, thus enhancing their suitability as medicaments.

In a first aspect of the present invention, there is provided a compound of Formula I,

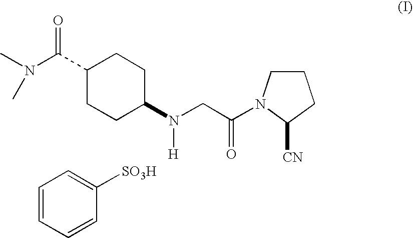

and anhydrate, hydrate or solvate forms thereof.

In one embodiment, the present invention provides a crystalline form of anhydrous trans-4-({2-[(2S)-2-cyanopyrrolidinyl]-2-oxoethyl}amino)-N,N-dim ethylcyclohexanecarboxamide besylate characterised by a powder x-ray diffraction pattern comprising the following peaks:

| Two theta (deg) | d-spacing (angstroms) |
|---|---|
| 6.1 ± 0.2 | 14.5 ± 0.5 |
| 14.1 ± 0.2 | 6.3 ± 0.1 |
| 16.3 ± 0.2 | 5.4 ± 0.1 |

More particularly, the crystalline form has a powder x-ray diffraction pattern that is substantially as shown in FIG. 1. The relative intensities of the diffraction peaks can vary due to variation in the preferred orientation of particles in the diffraction experiment sample preparation. The observed relative intensities are also influenced by the specific instrumental geometry of the powder diffractometer.

In another embodiment, the present invention provides a crystalline form of monohydrate trans-4-({2-[(2S)-2-cyanopyrrolidinyl]-2-oxoethyl}amino)-N,N-dim ethylcyclohexanecarboxamide besylate characterised by a powder x-ray diffraction pattern comprising the following peaks:

| Two theta (deg) | d-spacing (angstroms) |
|---|---|
| 6.8 ± 0.2 | 13.0 ± 0.4 |
| 7.8 ± 0.2 | 11.3 ± 0.3 |
| 8.8 ± 0.2 | 10.0 ± 0.3 |

More particularly, the crystalline form has a powder x-ray diffraction pattern that is substantially as shown in FIG. 2. The relative intensities of the diffraction peaks can vary due to variation in the preferred orientation of particles in the diffraction experiment sample preparation. The observed relative intensities are also influenced by the specific instrumental geometry of the powder diffractometer.

In a second aspect of the present invention, there is provided a pharmaceutical composition including a therapeutically effective amount of a compound of Formula I and anhydrate, hydrate or solvate forms thereof.

Furthermore, the present invention should be interpreted to include pharmaceutical compositions that include one or more anhydrate form of trans-4-({2-[(2S)-2-cyanopyrrolidinyl]-2-oxoethyl}amino)-N,N-dim ethylcyclohexanecarboxamide besylate, one or more hydrated form of trans-4-({2-[(2S)-2-cyanopyrrolidinyl]-2-oxoethyl}amino)-N,N-dim ethylcyclohexanecarboxamide besylate, and/or one or more solvated form of trans-4-({2-[(2S)-2-cyanopyrrolidinyl]-2-oxoethyl}amino)-N,N-dim ethylcyclohexanecarboxamide besylate.

Preferably, as used herein pharmaceutical compositions include one or more pharmaceutically acceptable carrier, diluent, or excipient.

In a third aspect of the present invention, there is provided a method of treating a disorder in a mammal having hyperglycemia or other conditions associated with diabetes, including: administering to said mammal a therapeutically effective amount of a compound of Formula I, and anhydrate, hydrate or solvate forms thereof.

In a fourth aspect of the present invention, there is provided a compound of Formula I and anhydrate, hydrate or solvate forms thereof, for use in medical therapy.

In a fifth aspect of the present invention, there is provided use of a compound of Formula I and anhydrate, hydrate or solvate forms thereof, in the preparation of a medicament for use in the treatment and/or prophylaxis of hyperglycemia or other conditions associated with diabetes.

It will be appreciated by the skilled person that a ‘benzenesulfonic acid’ salt may also be referred to as a ‘benzenesulfonate’ or ‘besylate’ salt.

### BRIEF DESCRIPTION OF THE DRAWINGS

FIG. 1 depicts the powder X-ray diffraction pattern (PXRD) of trans-4-({2-[(2S)-2-cyanopyrrolidinyl]-2-oxoethyl}-amino)-N,N-dimethylcyclohexanecarboxamide besylate anhydrate, using a conventional powder X-ray diffractometer with Bragg-Brentano geometry and copper K alpha radiation.

FIG. 2 depicts the powder X-ray diffraction pattern (PXRD) of trans-4-({2-[(2S)-2-cyanopyrrolidinyl]-2-oxoethyl}-amino)-N,N-dimethylcyclohexanecarboxamide besylate monohydrate, using a conventional powder X-ray diffractometer with Bragg-Brentano geometry and copper K alpha radiation.

FIG. 2a depicts the simulated powder X-ray diffraction pattern (PXRD) of trans-4-({2-[(2S)-2-cyanopyrrolidinyl]-2-oxoethyl}amino)-N,N-dimethylcyclohexanecarboxamide besylate monohydrate with copper K-alpha radiation.

FIGS. 3 (a) and (b) depict water sorption curves of (a) trans-4-({2-[(2S)-2-cyanopyrrolidinyl]-2-oxoethyl}amino)-N,N-dim ethylcyclohexanecarboxamide besylate monohydrate and (b) trans 4-({2-[(2S)-2-cyanopyrrolidinyl]-2-oxoethyl}-amino)-N,N-dimethylcyclohexanecarboxamide besylate anhydrate.

FIG. 4 depicts infrared (IR) studies relating to hydration/dehydration of trans-4-({2-[(2S)-2-cyano-pyrrolidinyl]-2-oxoethyl}amino)-N,N-dimethylcyclohexanecarboxamide besylate monohydrate.

FIG. 5 depicts the Raman spectra of trans-4-({2-[(2S)-2-cyanopyrrolidinyl]-2-oxoethyl}amino)-N,N-dimethylcyclohexanecarboxamide besylate monohydrate.

FIG. 6 depicts the Raman spectra of trans-4-({2-[(2S)-2-cyanopyrrolidinyl]-2-oxoethyl}amino)-N,N-dimethylcyclohexanecarboxamide besylate anhydrate.

FIG. 7 depicts the Raman spectra of the ethanol solvate of trans-4-({2-[(2S)-2-cyanopyrrolidinyl]-2-oxoethyl}amino)-N,N-dim ethylcyclohexanecarboxamide besylate.

FIG. 8 depicts the Raman spectra of the 1-propanol solvate of trans-4-({2-[(2S)-2-cyanopyrrolidinyl]-2-oxoethyl}-amino)-N,N-dimethylcyclohexanecarboxamide besylate.

FIG. 9 depicts the Raman spectra of the 2-propanol solvate of trans-4-({2-[(2S)-2-cyanopyrrolidinyl]-2-oxoethyl}-amino)-N,N-dimethylcyclohexanecarboxamide besylate.

FIG. 10 depicts the Raman spectra of the 2-methyl-1-propanol solvate of trans-4-({2-[(2S)-2-cyanopyrrolidinyl]-2-oxoethyl}amino)-N,N-dimethylcyclohexanecarboxamide besylate.

FIG. 11 depicts the Raman spectra of the acetone solvate of trans-4-({2-[(2S)-2-cyanopyrrolidinyl]-2-oxoethyl}amino)-N,N-dim ethylcyclohexanecarboxamide besylate.

FIG. 12 depicts the powder X-ray diffraction pattern (PXRD) of trans-4-({2-[(2S)-2-cyanopyrrolidinyl]-2-oxoethyl}-amino)-N,N-dimethylcyclohexanecarboxamide hydrochloride.

### DETAILED DESCRIPTION OF THE INVENTION

As discussed and illustrated throughout, the present invention includes certain solid state crystalline forms. Several methods for characterizing such forms exist, and the invention should not be limited by the methods chosen or the instrumentation used in characterizing the compounds of the present invention. For example, with regard to x-ray diffraction patterns, the diffraction peak intensities in the experimental patterns can vary, as is known in the art, primarily due to preferred orientation (non-random orientation of the crystals) in the prepared sample. As such, the scope of the present invention must be considered in light of the variability of characterization that is appreciated by those skilled in the art.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the terms “anhydrous” and “anhydrate” are used interchangeably. Likewise the terms “hydrous” and “hydrate” are used interchangeably.

Novel benzenesulfonic acid salts of trans-4-({2-[(2S)-2-cyanopyrrolidinyl]-2-oxoethyl}amino)-N,N-dimethylcyclohexanecarboxamide, which are suitable as serine protease inhibitors, have moisture sorption properties superior to other crystalline salts of trans-4-({2-[(2S)-2-cyanopyrrolidinyl]-2-oxo-ethyl}amino)-N,N-dimethylcyclohexanecarboxamide disclosed in the art. This is exemplified by the hygroscopicity of the crystalline hydrochloride (HCl) salt of trans-4-({2-[(2S)-2-cyanopyrrolidinyl]-2-oxoethyl}amino)-N,N-dimethylcyclohexanecarboxamide. The crystalline HCl salt of trans-4-({2-[(2S)-2-cyanopyrrolidinyl]-2-oxoethyl}amino)-N,N-dim ethylcyclohexanecarboxamide has been observed to deliquesce at 25° C. at relative humidities greater than 75% in approximately 20 hours. Additionally, this crystalline HCl salt was found to deliquesce at 40° C. at relative humidities of greater than 75% in approximately 24 hours. Comparatively, the benzenesulfonic acid salts of trans-4-({2-[(2S)-2-cyano-pyrrolidinyl]-2-oxoethyl}amino)-N,N-dimethylcyclohexanecarboxamide have not been observed to deliquesce under these same conditions. That is, the benzenesulfonic acid salts of the present invention sorb much lower amounts of water when exposed to a broad range of humidities and can be prepared in a physically stable crystal form, thus enhancing their suitability as medicaments.

Benzenesulfonic acids of Formula I have been observed to exist in a monohydrate form, designated Form 1, and an anhydrate form, designated Form 2. The monohydrate of the compound of Formula I, Form 1, has a theoretical water content of 3.73% w/w. Additionally, the monohydrate sorbs another 1-2% w/w water up to 95% relative humidity. Infrared (IR) studies have shown that the monohydrate loses water readily at room temperature when purged with dried air or nitrogen and re-hydration occurs rapidly upon exposure of the dehydrated form to the atmospheric humidity. It has also been observed that the anhydrate form converts to the hydrate in the presence of water vapour, such as at elevated relative humidity.

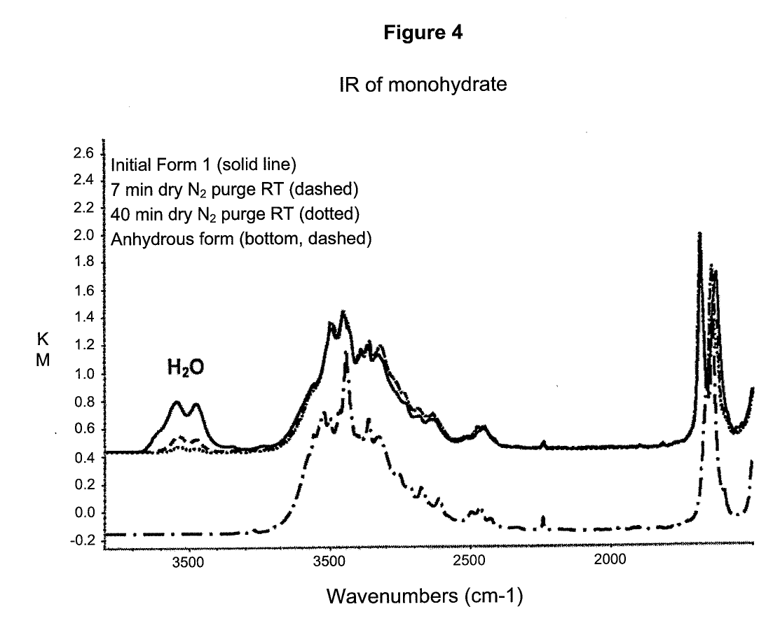

Additional infrared (IR) studies (FIG. 4) have shown that when the monohydrate Form 1 of Formula I is exposed to dry nitrogen for a given period of time, partial dehydration of the hydrate is facile and can occur under these low relative humidity conditions at room temperature. When exposed to higher relative humidities, re-hydration to the monohydrate occurs very rapidly. The partially dehydrated form of the monohydrate Form 1 of Formula I is stable for at least 24 hours at room temperature and partial hydration to the monohydrate of Formula I can be recovered at higher relative humidity (RH).

Importantly, although the above-referenced water contents are noted, the water content should not be considered as descriptive of any particular pharmaceutical composition or formulation comprising the forms of the present invention. Rather, when in admixture with other pharmaceutically acceptable carriers, diluents, or excipients, the water content may be higher or lower. The water contents given above should be considered as descriptive of the specific forms, themselves.

As examples of the preferred compounds of the present invention, anhydrous Form 2 may be characterized by, among other properties, a melting point of about 157° C. Likewise monohydrate Form 1 may be characterized by, among other properties, a melting point which occurs between 110 to 120° C.

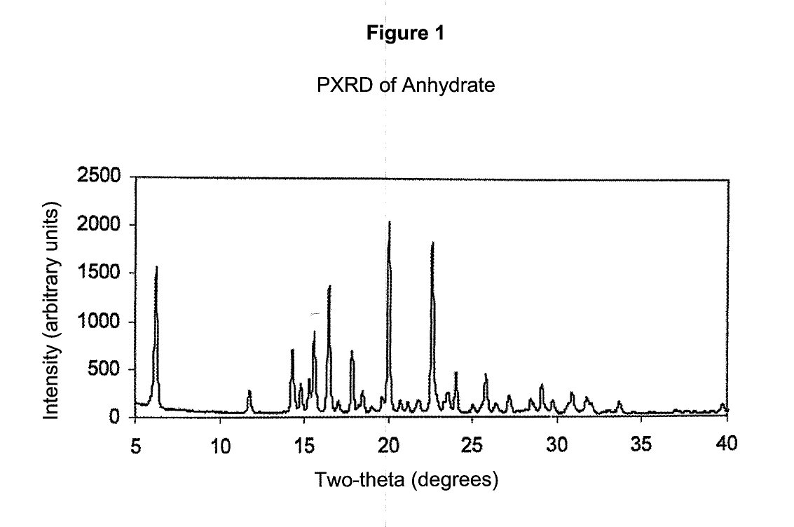

In one embodiment, the compound is the anhydrate Form 2 of the compound of Formula I characterized, in part, by a powder x-ray diffraction pattern as shown in FIG. 1. The anhydrate Form 2 of the compound of Formula I may be characterised by including, but not limited to, the peaks of Table I.

TABLE I
(Form 2 anhydrate)

| Two theta (deg) * | d-spacing (angstroms) |
|---|---|
| 6.1 ± 0.2 | 14.5 ± 0.5 |
| 14.1 ± 0.2 | 6.3 ± 0.1 |
| 16.3 ± 0.2 | 5.4 ± 0.1 |
| 19.8 ± 0.2 | 4.5 ± 0.1 |
| 22.4 ± 0.2 | 4.0 ± 0.1 |

* Based on Cu Kα radiation. Kα2 was removed prior to peak location

Notably, in a mixture of the anhydrate Form 2 of the compound of Formula I with another phase, not all the peaks listed in Table I may be apparent in the mixture's powder diffraction pattern.

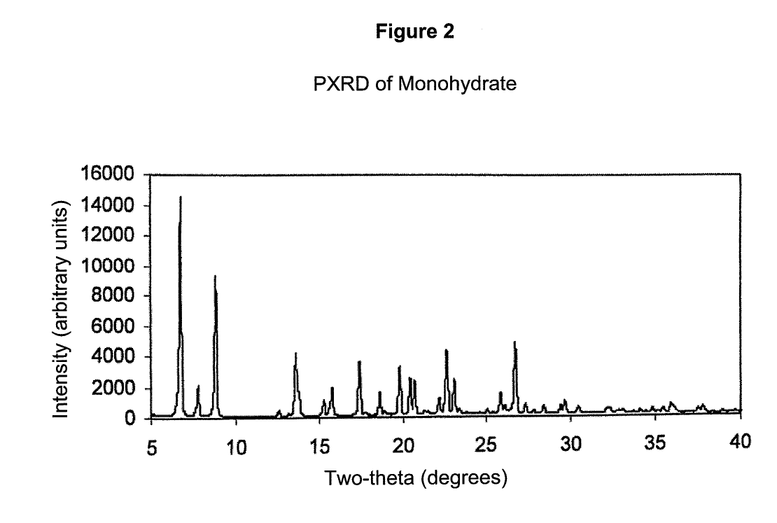

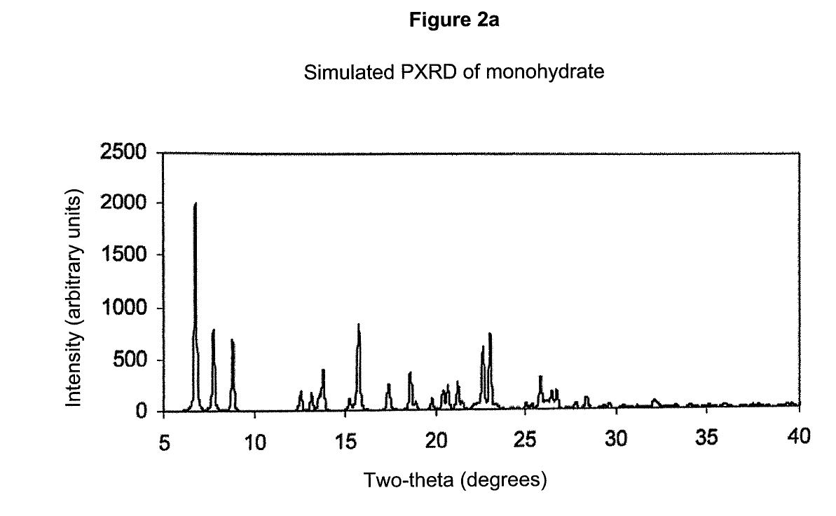

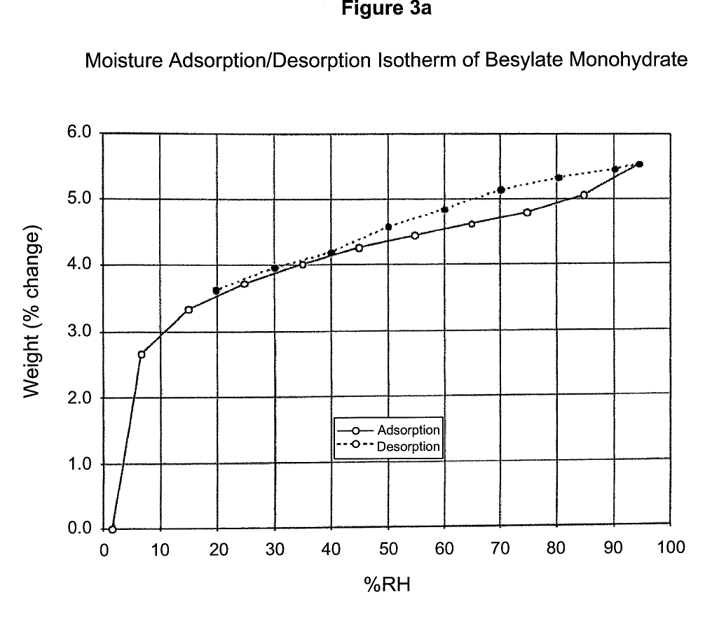

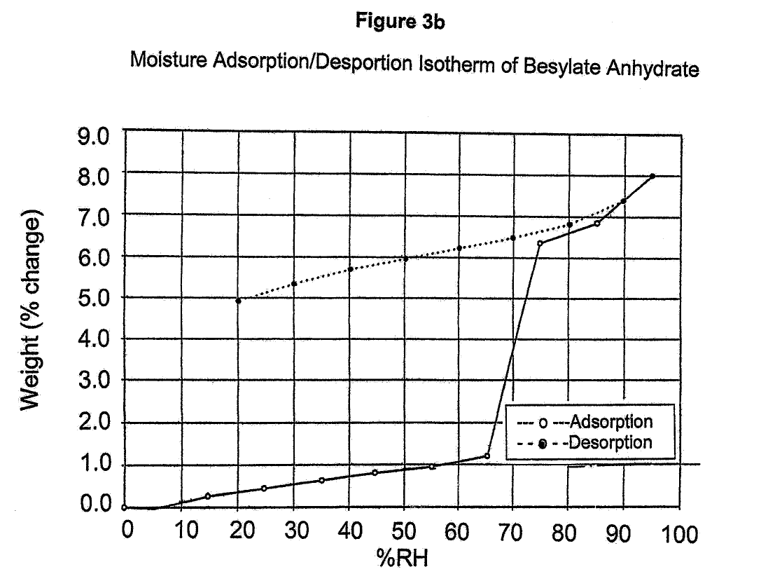

In another embodiment, the compound is the monohydrate Form 1 of the compound of Formula I characterized, in part, by a powder x-ray diffraction pattern as shown in FIG. 2. The monohydrate Form 1 of the compound of Formula I may be characterised by including, but not limited to, the peaks of Table II.

TABLE II
(Form 1 monohydrate)

| Two theta (deg) * | d-spacing (angstroms) |
|---|---|
| 6.8 ± 0.2 | 13.0 ± 0.4 |
| 7.8 ± 0.2 | 11.3 ± 0.3 |
| 8.8 ± 0.2 | 10.0 ± 0.3 |
| 17.4 ± 0.2 | 5.1 ± 0.1 |
| 26.7 ± 0.2 | 3.3 ± 0.1 |

* Based on Cu Kα radiation. Kα2 was removed prior to peak location

Notably, in a mixture of the monohydrate Form 1 of the compound of Formula I with another phase, not all the peaks listed in Table II may be apparent in the mixture's powder diffraction pattern.

The compounds of Formula I include within their scope substantially pure anhydrate, hydrate or solvate forms, as well as mixtures of solvate, hydrate and anhydrate forms. It is also understood, that such compounds include crystalline or amorphous forms and mixtures of crystalline and amorphous forms. The term ‘substantially pure’ means less than 10% of another form, preferably less than 5%, more preferably less than 1%, is present.

The free base and HCl salts of the compounds of Formula I may be prepared according to the procedures of the International Patent Application publication WO02/30891, referred to above.

As illustrated in Scheme A, the compound of Formula I, i.e., trans-4-({2-[(2S)-2-cyanopyrrolidinyl]-2-oxoethyl}amino)-N,N-dim ethylcyclohexanecarboxamide besylate has been prepared in two distinct forms, a monohydrate form (Form 1) (Formula I′ in Scheme A) and an anhydrate form (Form 2) (Formula I″ in Scheme A). The relationship of these forms is illustrated in Scheme B below.

### Anhydrate Form 2

The anhydrate Form 2 of trans-4-({2-[(2S)-2-cyanopyrrolidinyl]-2-oxoethyl}amino)-N,N-dim ethylcyclohexanecarboxamide besylate may be prepared by (a) reacting (2S)-1-(chloroacetyl)-2-pyrrolidinecarbonitrile with trans-4-amino-N,N-dimethylcyclohexane-carboxamide in acetonitrile in the presence of a base, such as potassium carbonate, followed by (b) introducing a dilute acid solution, such as a solution of citric acid and an extraction solvent such as dichloromethane and separating the layers, (c) adding 5N sodium hydroxide to adjust the pH to within a range of 8-11 to the aqueous layer and adding an extraction solvent such as dichloromethane, (d) separating the organic phase, and then (e) solvent exchanging the dichloromethane for a higher boiling temperature solvent such as methyl acetate (f) adding benzenesulfonic acid hydrate to the solution (g) and distilling the azeotrope of methyl acetate, for example, and water to provide the besylate anhydrate. Interconversion to the monohydrate and back to the anhydrate of the besylate salt compounds of the invention is as depicted in Scheme B.

### Monohydrate Form 1

The monohydrate Form 1 of trans-4-({2-[(2S)-2-cyanopyrrolidinyl]-2-oxoethyl}amino)-N,N-dim ethylcyclohexanecarboxamide besylate may be prepared by (a) reacting (2S)-1-(chloroacetyl)-2-pyrrolidinecarbonitrile with trans-4-amino-N,N-dimethylcyclohexane-carboxamide in acetonitrile in the presence of a base, such as potassium carbonate, followed by (b) introducing an dilute acid solution, such as a solution of citric acid and an extraction solvent such as dichloromethane and separating the layers, (c) adding 5N sodium hydroxide to adjust the pH to within a range of 8-11 to the aqueous layer and adding an extraction solvent such as dichloromethane, (d) separating the organic phase, and then (e) solvent exchanging the dichloromethane for a higher boiling temperature solvent such as 2-butanone (f) adding water to the 2-butanone solution (g) and adding benzenesulfonic acid hydrate to the solution to provide the besylate monohydrate.

Thus, the anhydrate form of the compound of formula I is obtainable by reaction of trans-4-({2-[(2S)-2-cyanopyrrolidinyl]-2-oxoethyl}amino)-N,N-dim ethylcyclohexanecarboxamide with benzenesulphonic acid and crystallisation in a polar solvent, e.g. methyl acetate (preferably anhydrous methyl acetate), and the monohydrate form of the compound of formula I is obtainable by reaction of trans-4-({2-[(2S)-2-cyanopyrrolidinyl]-2-oxoethyl}amino)-N,N-dim ethylcyclohexanecarboxamide with benzenesulphonic acid and crystallisation in a mixture of a polar solvent, e.g. 2-butanone, and water.

In another aspect, the present invention provides a process for preparing a compound of Formula I by reaction of trans-4-({2-[(2S)-2-cyanopyrrolidinyl]-2-oxoethyl}amino)-N,N-dim ethylcyclohexanecarboxamide with benzenesulfonic acid followed by crystallisation in a polar solvent, e.g. methyl acetate.

In another aspect, the present invention provides a process for preparing a compound of Formula I by reaction of trans-4-({2-[(2S)-2-cyanopyrrolidinyl]-2-oxoethyl}amino)-N,N-dim ethylcyclohexanecarboxamide with benzenesulfonic acid followed by crystallisation in a mixture of a polar solvent, e.g. 2-butanone, and water.

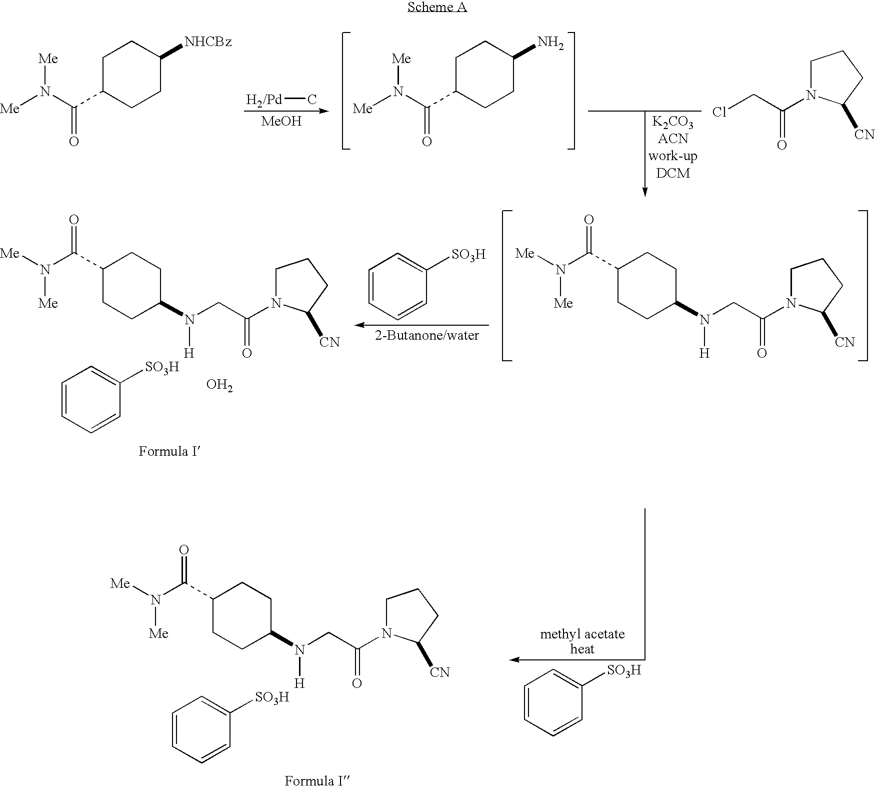

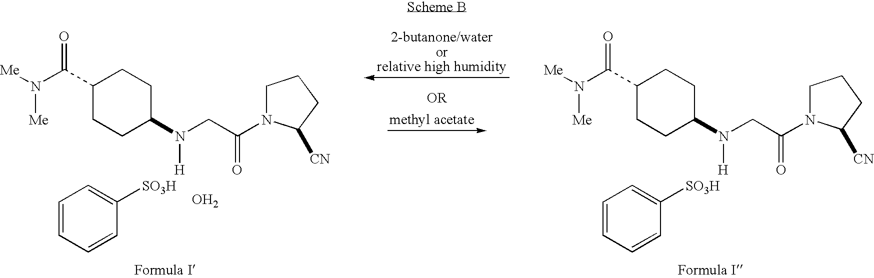

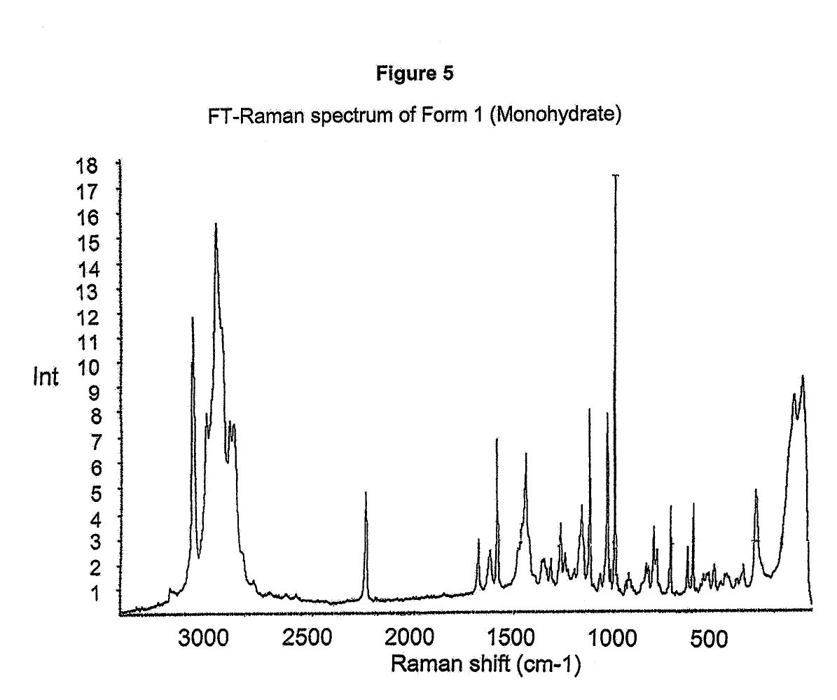

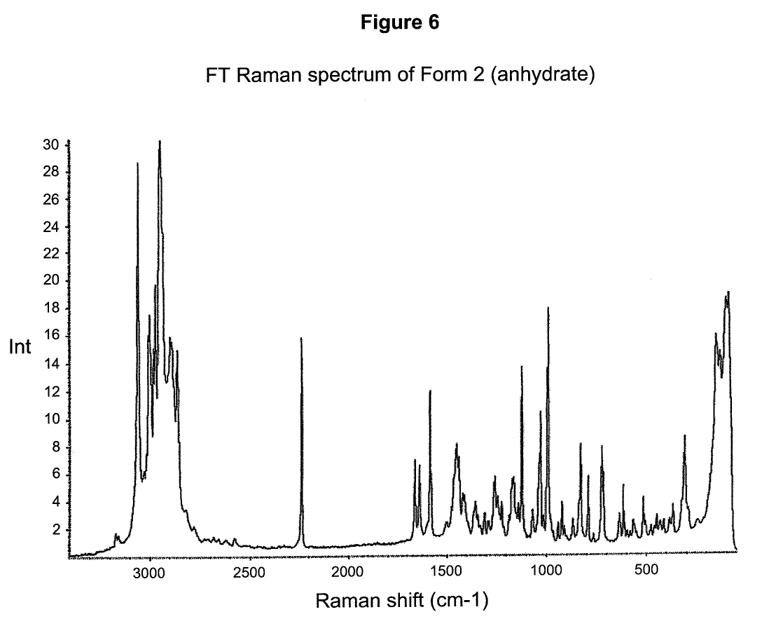

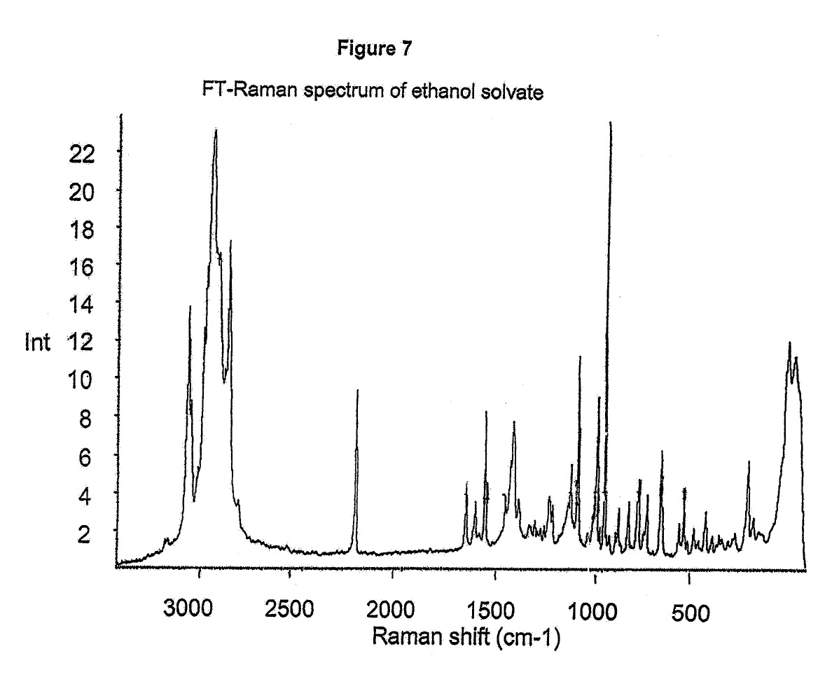

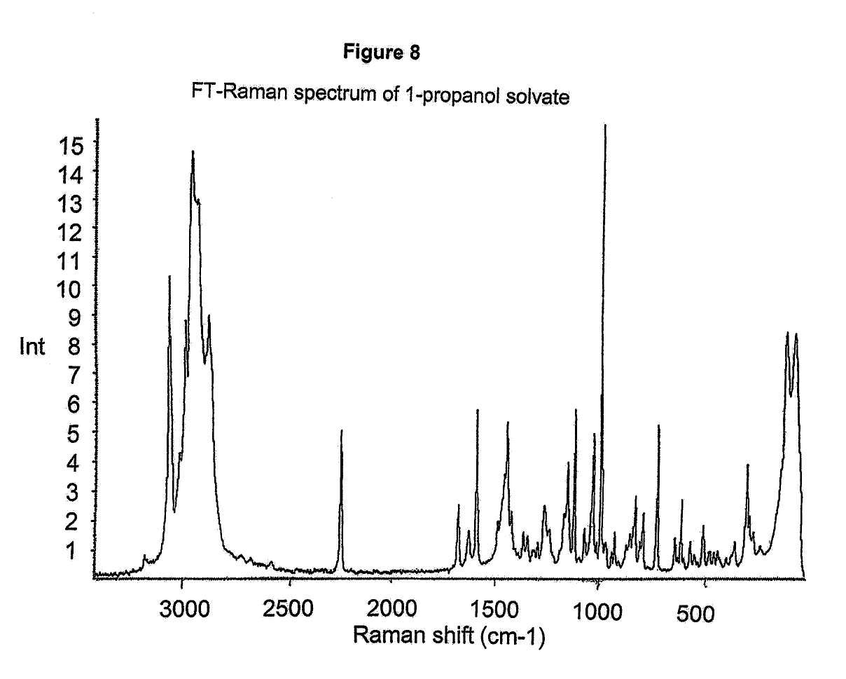

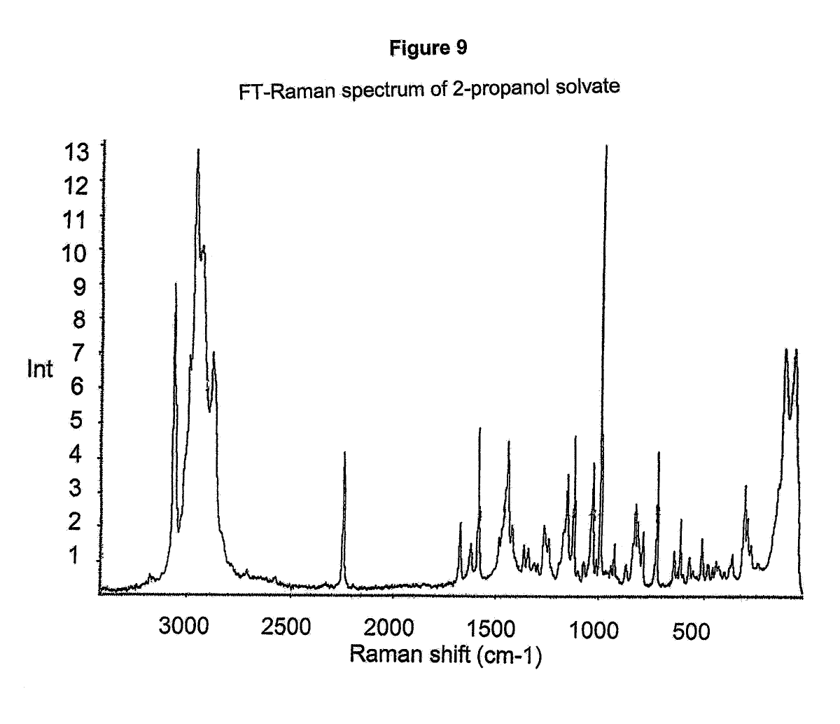

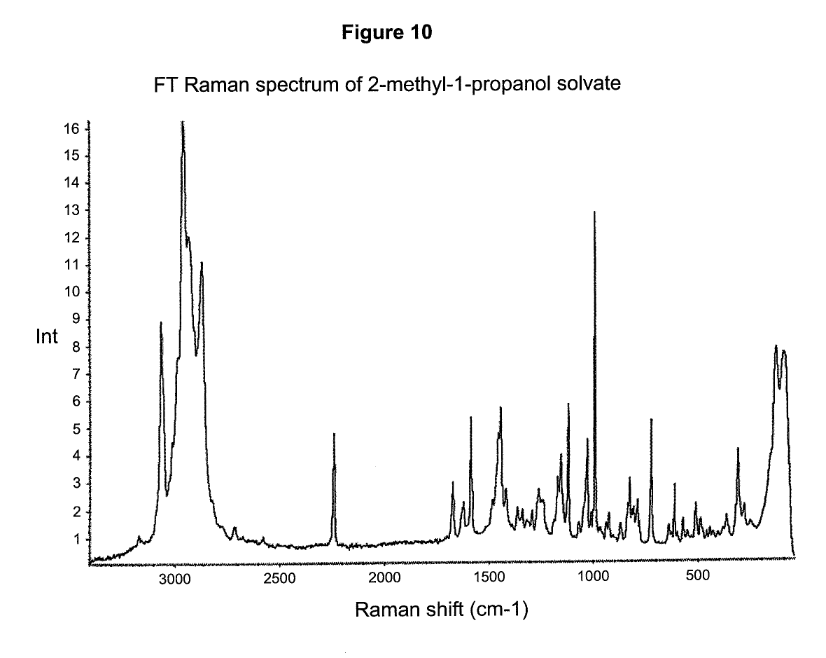

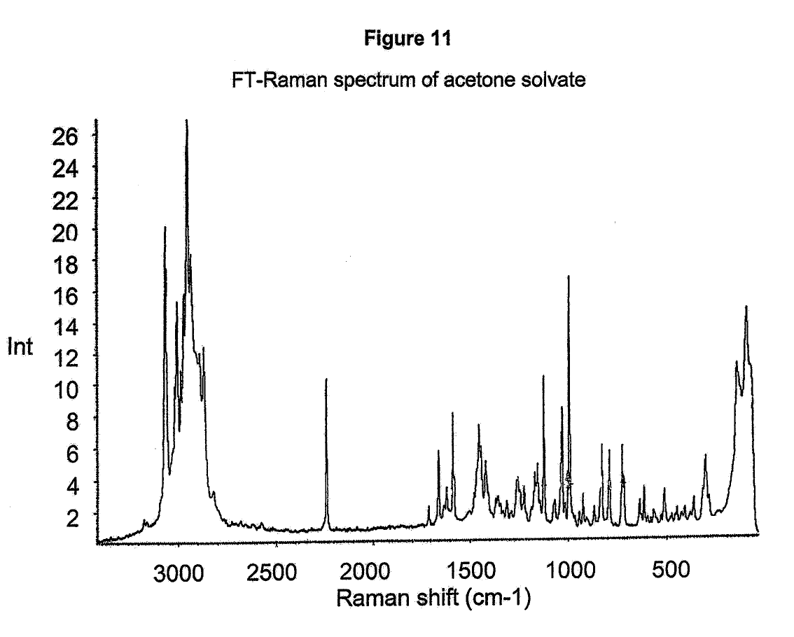

A series of crystallization experiments were performed to investigate whether the benzenesulfonic acid salt of Formula I can either exist in more than one solid-state form or has a propensity to form solvates. This series of experiments employed 45 different solvent systems and four crystallization modes (slow evaporation, fast evaporation, cooling, and ripening). These experiments indicated the existence of at least two non-solvated solid-state forms, the monohydrate and the anhydrate of the compound of Formula I. Additionally, benzenesulfonic acid salts of Formula I were shown to form solvates in the presence of several hydrogen-bond-donor solvents for example ethanol, 1-propanol, 2-propanol, 2-methyl-1-propanol and acetone. The Raman spectra of solvates of benzenesulfonic acid salts of Formula I are shown in FIGS. 5-11.

While it is possible that, for use in therapy, therapeutically effective amounts of a compound of Formula I, as well as anhydrate or hydrate forms thereof, may be administered as the raw chemical, it is possible to present the active ingredient as a pharmaceutical composition. Accordingly, the invention further provides pharmaceutical compositions which include therapeutically effective amounts of compounds of the Formula I and anhydrate or hydrate forms thereof, and one or more pharmaceutically acceptable carriers, diluents, or excipients. The compounds of the Formula I and anhydrate or hydrate forms thereof, are as described above. The carrier(s), diluent(s) or excipient(s) must be acceptable in the sense of being compatible with the other ingredients of the formulation and not deleterious to the recipient thereof. According to another aspect of the invention there is also provided a process for the preparation of a pharmaceutical formulation including admixing a compound of the Formula I, or anhydrate, hydrate or solvate forms thereof, with one or more pharmaceutically acceptable carriers, diluents or excipients.

Compounds of Formula I and anhydrate, hydrate or solvate forms thereof may be formulated for administration by any route, and the appropriate route will depend on the disease being treated as well as the subjects to be treated. Suitable pharmaceutical formulations include those for oral, rectal, nasal, topical (including buccal, sub-lingual, and transdermal), vaginal or parenteral (including intramuscular, sub-cutaneous, intravenous, and directly into the affected tissue) administration or in a form suitable for administration by inhalation or insufflation. The formulations may, where appropriate, be conveniently presented in discrete dosage units and may be prepared by any of the methods well know in the pharmacy art.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodiumbenzoate, sodiumacetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compound of Formula I and anhydrate, hydrate or solvate forms thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of Formula I and anhydrate, hydrate or solvate forms thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamidephenol, polyhydroxyethylaspartamide-phenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

Also provided in the present invention, is a method for inhibiting a post proline/analine cleaving protease, such as a serine protease, such as a dipeptidyl peptidase, such as DPP-IV, which includes administering a therapeutically effective amount of a compound of the present invention including anhydrate, hydrate or solvate forms thereof, to the mammal.

A therapeutically effective amount of a compound of Formula I and anhydrate, hydrate or solvate forms thereof will depend on a number of factors including, but not limited to, the age and weight of the mammal, the precise disorder requiring treatment and its severity, the nature of the formulation, and the route of administration, and will ultimately be at the discretion of the attendant physician or veterinarian. Typically, the compound of Formula I and anhydrate, hydrate or solvate forms thereof will be given for treatment in the range of 0.1 to 100 mg/kg body weight of recipient (mammal) per day and more usually in the range of 1 to 10 mg/kg body weight per day. Acceptable daily dosages, may be from about 0.1 to about 1000 mg/day, and preferably from about 0.1 to about 100 mg/day.

The compounds of the present invention including anhydrate, hydrate and/or solvate forms thereof, described above, are useful in therapy and in the preparation of medicaments for treating a disorder in a mammal, which is characterized by the need for inhibition of a post proline/analine cleaving protease, such as a serine protease, such as a dipeptidyl peptidase, such as DPP IV. The compounds of the present invention including anhydrate, hydrate and/or solvate forms thereof are useful for treating or preventing metabolic disorders, gastrointestinal disorders, viral disorders, autoimmune disorders, dermatological or mucous membrane disorders, compliment mediated disorders, inflammatory disorders, and psychosomatic, depressive, and neuropsychiatric disorders, including, without limitation, diabetes, obesity, hyperlipidemia, psoriasis, intestinal distress, constipation, encephalomyelitis, glumerulonepritis, lipodystrophy, tissue damage, HIV infection, allergies, inflammation, arthritis, transplant rejection, high blood pressure, congestive heart failure, tumors, and stress-induced abortions.

The compound of Formula I and anhydrate, hydrate or solvate forms thereof, described above, are useful in therapy and in the preparation of a medicament for the treatment and/or prophylaxis of hyperglycemia or other conditions associated with diabetes.

The following examples are intended for illustration only and are not intended to limit the scope of the invention in any way.

### EXAMPLE 1

### Preparation of trans-4-({2-[(2S)-2-cyanopyrrolidinyl]-2-oxoethyl}amino)-N,N-dimethylcyclohexanecarboxamide benzenesulfonate monohydrate

### Preparation of trans-4-amino-N,N-dimethylcyclohexanecarboxamide

To a solution of benzyl trans-4-[(dimethylamino)-carbonyl]cyclohexylcarbamate (1 wt., 1 eq) in methanol (7 vol) was added palladium on carbon (0.1 wt). The reaction was hydrogenated under 0.2 bar over atmosphere of hydrogen until the reaction was complete (monitored by react IR). After purging the reaction with nitrogen, the reaction was filtered to remove the catalyst, and rinsed with methanol (2 vol). The filtrate was solvent exchanged to acetonitrile (10 vol). This solution of 4-amino-N,N-dimethylcyclohexanecarboxamide was used directly in the next step.

### Preparation of trans-4-({2-[(2S)-2-cyanopyrrolidinyl]-2-oxoethyl}amino)-N,N-dimethylcyclohexanecarboxamide

To a solution of trans-4-amino-N,N-dimethylcyclohexanecarboxamide (1 wt., 1 eq.) in acetonitrile (10 vol) was added potassium carbonate (2.3 wt., 2.8 eq.) followed by (2S)-1-(chloroacetyl)-2-pyrrolidinecarbonitrile (0.96 wt., 0.95 eq.). The resulting slurry was stirred at approx. 25° C. for 20-24 hours. The reaction was filtered, washed with acetonitrile (1 vol), and the filtrate was concentrated to minimum volume under vacuum. Dichloromethane (5 vol) and 10% aqueous citric acid (5 vol) were added and the biphasic mixture was stirred for 10 minutes. The layers were separated and to the aqueous layer was added dichloromethane (5 vol) The biphasic mixture was cooled to approx. 5° C. and 50% aqueous potassium carbonate (2.3 vol) was added while keeping the temperature at approx. 5° C. The layers were separated and the organic layer was washed with brine (2 vol.). The organic layer (dichloromethane solution of trans-4-({2-[(2S)-2-cyano-pyrrolidinyl]-2-oxoethyl}amino)-N,N-dimethylcyclohexanecarboxamide) was used directly in the next step.

### Preparation of trans-4-({2-[(2S)-2-cyanopyrrolidinyl]-2-oxoethyl}amino)-N,N-dimethylcyclohexanecarboxamide besylate monohydrate

The crude methylene chloride solution from above was charged to the reaction vessel. Solvent exchange with (8 volumes) of 2-butanone removing excess DCM under reduced pressure while maintaining the internal temperature at or below 25° C. To the 2-butanone solution is added 0.11 wt (2 eq) of process water. A solution of benzenesulfonic acid was prepared by dissolving 0.50 wt in 2.5 volumes of 2-butanone. Approximately 10% of this solution was added slowly. Seed crystals were then introduced (0.5 wt %). The remainder of the acid solution was then added dropwise. After completion of the addition, the suspension was stirred at high speed for 1 hr at 25° C. The precipitate was then collected on Whatman no. 1 filter paper in a Buchner funnel. The cake was washed successively with 2×2 volumes of 2-butanone and then suction/air dried. The damp cake was then transferred to a crystallization dish and placed in a vacuum oven (70° C., house vacuum, N₂ bleed)

Expected yield: 80-95%

### EXAMPLE 2

### Powder X-Ray Diffraction of Monohydrate Besylate Salt

The monohydrate besylate salt prepared according to Example 1 was prepared by placing the sample on to a silicon zero background plate and scanning with a conventional Bragg-Brentano diffractometer with copper K alpha radiation at ambient room temperature. The powder X-ray diffraction pattern obtained is shown in FIG. 2.

### EXAMPLE 3

### Single Crystal X-Ray Diffraction of Monohydrate Besylate Salt

The single crystal structure of the monohydrate besylate salt was determined using copper K-alpha radiation at 293K. The crystal system, space group, and cell parameters are provided:

### Crystal System Monoclinic

### Space Group: P2₁

### Lattice Parameters

a=11.785(1) Å
b=8.367(1) Å
c=13.541(1) Å
β=105.657(4) Å

### V=1285.7(2) Å³

The simulated powder X-ray diffraction pattern using copper K-alpha radiation is shown in FIG. 2a. The differences between the simulated pattern and the experimental monohydrate pattern in FIG. 2 are primarily due to preferred orientation in the experimental pattern, minor differences in lattice parameters between single crystal and experimental powder data, specific peak profile parameters used in the simulated pattern calculation, and to a lesser extent due to differences in atomic disorder between the experimental powder and single crystal data.

### EXAMPLE 4

### Preparation of trans-4-({2-[(2S)-2-cyanopyrrolidinyl]-2-oxoethyl}amino)-N,N-dimethylcyclohexanecarboxamide besylate anhydrate

A solution of trans-4-({2-[(2S)-2-cyanopyrrolidinyl]-2-oxoethyl}amino)-N,N-dim ethylcyclohexane-carboxamide (1000 mg) in 8 mL of anhydrous methyl acetate was warmed to 45° C. In a separate vessel, 527 mg (1 eq.) of anhydrous benzene sulfonic acid was dissolved in 2.5 mL of anhydrous methyl acetate. The solution of benzene sulfonic acid was then added dropwise to the warm methyl acetate solution described above. When the addition was complete, the mixture was stirred at 45° C. for an additional 40 minutes and then allowed to cool naturally to room temperature. The solids were then collected on Whatman no. 2 filter paper under a N₂ blanket to give 987 mg (65% th) of trans-4-({2-[(2S)-2-cyanopyrrolidinyl]-2-oxoethyl}amino)-N,N-dim ethylcyclohexanecarboxamide besylate anhydrate.

### EXAMPLE 5

### Powder X-Ray Diffraction of Anhydrate Besylate Salt

The anhydrate besylate salt prepared according to Example 4 was prepared by placing the sample on to a silicon zero background plate and scanning with a conventional Bragg-Brentano diffractometer with copper K alpha radiation at ambient room temperature. The powder X-ray diffraction pattern obtained is shown in FIG. 1.

### EXAMPLE 6

### Infrared (IR) Studies of trans-4-({2-[(2S)-2-cyano-pyrrolidinyl]-2-oxoethyl}amino)-N,N-dimethylcyclohexanecarboxamide besylate

Samples of trans-4-({2-[(2S)-2-cyanopyrrolidinyl]-2-oxoethyl}amino)-N,N-dimethylcyclohexane-carboxamide besylate monohydrate either as a pure solid or diluted in KBr powder were placed inside a FTIR spectrometer purged under dry N₂ at room temperature (RT). IR spectra recorded at different times showed that the intensities of IR bands at 3544.8, 3471.5 and 1626.8 cm⁻¹, which can be assigned to H₂O, decreased very rapidly within 10 min. and reached an equilibrium in ˜30 min. Upon re-exposure of the partially dehydrated samples to the atmosphere for <1 min., the intensities of these IR bands are fully recovered. The FTIR spectrum of this experiment is in FIG. 4.

### EXAMPLE 7

### Moisture Sorption Testing 4-({2-[(2S)-2-cyanopyrrolidinyl]-2-oxoethyl}amino)-N,N-dimethylcyclohexanecarboxamide besylate monohydrate salt

Moisture sorption was measured with an integrated gas flow microbalance system. Approximately 20 mg of 4-({2-[(2S)-2-cyanopyrrolidinyl]-2-oxoethyl}amino)-N,N-dimethylcyclohexanecarboxamide besylate monohydrate salt was weighed into a sample pan of an integrated gas flow microbalance system (SGA100, VTI Corporation). The sample was dried at 60° C. under a dry nitrogen stream until the rate of weight loss was less than 0.015% in 5 minutes. After drying the sample was equilibrated at 25° C. and the relative humidity increased stepwise (adsorption) to 5, 15, 25, 35, 45, 55, 65, 75, 85 and 95% Each relative humidity step was held until the sample equilibrated at that condition. Equilibrium was defined as a weight change of less than 0.015% in 5 minutes. The relative humidity was then decreased step wise (desorption) to 90, 80, 70, 60, 50, 40, and 20% Each step was held until equilibrium was reached. The equilibrium condition was the same as in the sorption phase. The % w/w increase or decrease in moisture content of the sample is reported for each equilibrated RH condition.

Typically, the monohydrate form absorbs less than 10% w/w water, preferably less than 8% w/w water, more preferably less than 6% w/w water at relative humidity between 0% and 95% at 25° C. Water adsorbed by monohydrate Form 1 readily desorbs when the relative humidity is decreased.

### EXAMPLE 8

Moisture sorption testing 4-({2-[(2S)-2-cyanopyrrolidinyl]-2-oxoethyl}amino)-N,N-dimethylcyclohexanecarboxamide besylate anhydrate salt

Moisture sorption was measured with an integrated vacuum microbalance system. Approximately 11 mg of 4-({2-[(2S)-2-cyanopyrrolidinyl]-2-oxoethyl}amino)-N,N-dimethylcyclohexanecarboxamide besylate anhydrate salt was weighed into a sample pan of an integrated vacuum microbalance system (MB300G, VTI Corporation). The sample was dried at 60° C. under vacuum until the rate of weight loss was less than 0.015% in 2 minutes. After drying the sample was equilibrated at 25° C. and the relative humidity increased stepwise (adsorption) to 15, 25, 35, 45, 55, 65, 75, 85 and 95% Each relative humidity step was held until the sample equilibrated at that condition. Equilibrium was defined as a weight change of less than 0.015% in 2 minutes. The relative humidity was then decreased step wise (desorption) to 90, 80, 70, 60, 50, 40, 30 and 20% Each step was held until equilibrium was reached. The equilibrium condition was the same as in the sorption phase. The % w/w increase or decrease in moisture content of the sample is reported for each equilibrated RH condition.

Typically, the anhydrate form converts to the hydrate between 65% and 75% relative humidity at 25° C.

### EXAMPLE 9

### Raman Spectra of 4-({2-[(2S)-2-cyanopyrrolidinyl]-2-oxoethyl}amino)-N,N-dimethylcyclohexanecarboxamide solvates In a typical process, 4-({2-[(2S)-2-cyanopyrrolidinyl]-2-oxoethyl}amino)-N,N-dimethylcyclohexanecarboxamide is suspended in ethanol in the presence of seed material and warmed to 60° C. for one hour. All Raman spectra were obtained on a Thermo Nicolet 960 FT Raman spectrometer equipped with a ViewStage with a spectral resolution of 4 cm⁻¹. FT Raman Spectra are given in FIGS. 5-11.

Below is a table summarizing several of the major peaks and their corresponding relative intensities. As noted hereinabove, due to the variability appreciated by those skilled in the art, the existence of peaks and intensities should not necessarily be interpreted too critically in the characterization of a compound within the scope of the present invention.

Raman Peak Table of Formula I Solvates

| | | | | 2-methyl-1- | | ace- |
|---|---|---|---|---|---|---|
| Form 1 | Form 2 | Ethanol | 1-propanol | propanol | 2-propanol | tone |
| 96 | 93 | 109 | 99 | 106 | 98 | 108 |
| 135 | 106 | 139 | 139 | 139 | 143 | 156 |
| 317 | 136 | 294 | 305 | 287 | 304 | 312 |
| 375 | 155 | 322 | 318 | 317 | 318 | 370 |
| 515 | 312 | 375 | 370 | 368 | 370 | 415 |
| 618 | 368 | 451 | 447 | 491 | 447 | 452 |
| 644 | 450 | 481 | 463 | 515 | 462 | 516 |
| 727 | 517 | 517 | 482 | 574 | 485 | 568 |
| 793 | 618 | 570 | 515 | 616 | 515 | 617 |
| 809 | 638 | 618 | 554 | 642 | 572 | 639 |
| 846 | 717 | 639 | 573 | 728 | 615 | 718 |
| 997 | 726 | 730 | 616 | 793 | 644 | 727 |
| 1018 | 793 | 793 | 644 | 810 | 728 | 793 |
| 1035 | 832 | 834 | 729 | 832 | 792 | 832 |
| 1122 | 870 | 881 | 792 | 874 | 820 | 870 |
| 1163 | 925 | 929 | 832 | 929 | 831 | 927 |
| 1246 | 993 | 941 | 855 | 998 | 872 | 998 |
| 1269 | 1000 | 973 | 929 | 1014 | 928 | 1033 |
| 1316 | 1018 | 997 | 943 | 1033 | 943 | 1071 |
| 1351 | 1032 | 1019 | 973 | 1072 | 996 | 1126 |
| 1443 | 1072 | 1036 | 997 | 1125 | 1013 | 1159 |
| 1482 | 1125 | 1129 | 1014 | 1158 | 1033 | 1174 |
| 1587 | 1143 | 1163 | 1034 | 1174 | 1074 | 1227 |
| 1622 | 1166 | 1251 | 1074 | 1265 | 1123 | 1259 |
| 1680 | 1227 | 1269 | 1124 | 1295 | 1157 | 1314 |
| 2240 | 1246 | 1291 | 1158 | 1342 | 1243 | 1359 |
| 2871 | 1261 | 1338 | 1268 | 1365 | 1268 | 1421 |
| 2891 | 1312 | 1366 | 1295 | 1423 | 1342 | 1443 |
| 2956 | 1357 | 1420 | 1343 | 1447 | 1364 | 1454 |
| 3003 | 1422 | 1450 | 1365 | 1588 | 1423 | 1588 |
| 3068 | 1442 | 1486 | 1423 | 1624 | 1445 | 1620 |
| | 1454 | 1588 | 1445 | 1675 | 1587 | 1662 |
| | 1587 | 1635 | 1588 | 2244 | 1620 | 1712 |
| | 1641 | 1681 | 1622 | 2717 | 1674 | 2240 |
| | 1664 | 2240 | 1675 | 2871 | 2246 | 2867 |
| | 2242 | 2870 | 2246 | 2935 | 2878 | 2888 |
| | 2864 | 2919 | 2877 | 2961 | 2929 | 2935 |
| | 2899 | 2949 | 2961 | 3065 | 2962 | 2952 |
| | 2947 | 2981 | 2988 | | 2989 | 2968 |
| | 2972 | 2994 | 3063 | | 3061 | 2984 |
| | 2982 | 3054 | | | | 3003 |
| | 3003 | 3066 | | | | 3016 |
| | 3060 | | | | | 3062 |

### EXAMPLE 10

Powder X-Ray Diffraction of 4-({2-[(2S)-2-cyanopyrrolidinyl]-2-oxoethyl}amino)-N,N-dimethylcyclohexanecarboxamide hydrochloride salt

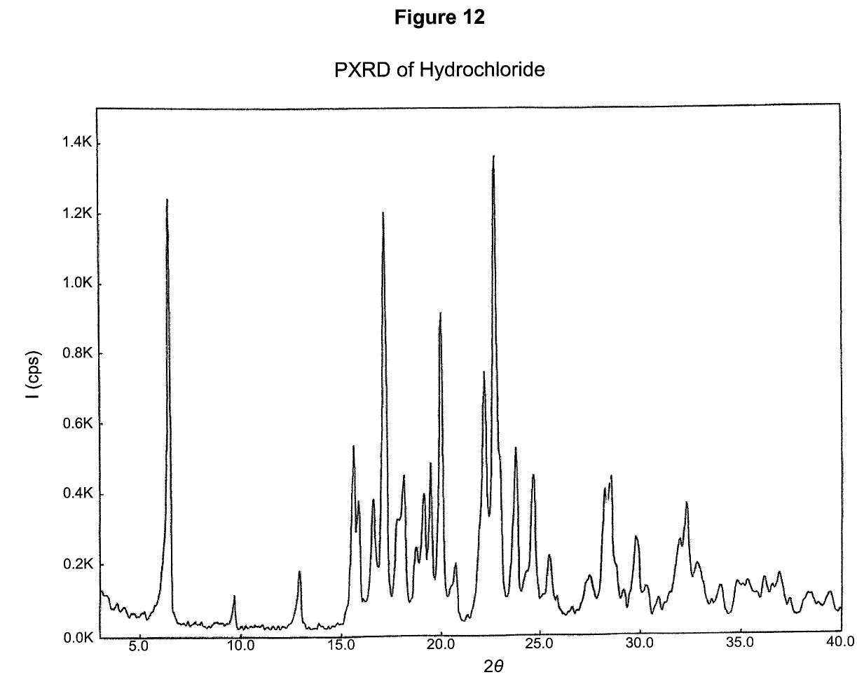

The powder X-ray diffraction pattern for 4-({2-[(2S)-2-cyanopyrrolidinyl]-2-oxoethyl}amino)-N,N-dimethylcyclohexanecarboxamide hydrochloride salt is shown in FIG. 12.

Below is a table summarizing the major peaks and their corresponding relative intensities.

| No. | 2Theta | d | I (cps) | I/Io | FWHM |
|---|---|---|---|---|---|
| 1 | 6.4 | 13.7 | 1243 | 940 | 0.16 |
| 2 | 9.7 | 9.1 | 110 | 65 | 0.16 |
| 3 | 12.9 | 6.8 | 177 | 120 | 0.16 |
| 4 | 15.6 | 5.7 | 534 | 375 | 0.18 |
| 5 | 15.9 | 5.6 | 376 | 245 | 0.14 |
| 6 | 16.6 | 5.3 | 380 | 233 | 0.20 |
| 7 | 17.2 | 5.2 | 1204 | 874 | 0.20 |
| 8 | 17.8 | 5.0 | 324 | 178 | 0.16 |
| 9 | 18.1 | 4.9 | 448 | 275 | 0.18 |
| 10 | 18.7 | 4.7 | 241 | 109 | 0.16 |
| 11 | 19.1 | 4.6 | 393 | 227 | 0.16 |
| 12 | 19.5 | 4.6 | 482 | 301 | 0.16 |
| 13 | 20.0 | 4.4 | 914 | 652 | 0.18 |
| 14 | 20.7 | 4.3 | 196 | 110 | 0.16 |
| 15 | 22.2 | 4.0 | 741 | 518 | 0.20 |
| 16 | 22.8 | 3.9 | 1366 | 1000 | 0.18 |
| 17 | 23.0 | 3.9 | 453 | 279 | 0.06 |
| 18 | 23.8 | 3.7 | 522 | 330 | 0.20 |
| 19 | 24.7 | 3.6 | 442 | 275 | 0.22 |
| 20 | 25.4 | 3.5 | 214 | 108 | 0.22 |
| 21 | 27.5 | 3.2 | 155 | 62 | 0.38 |
| 22 | 28.2 | 3.2 | 403 | 249 | 0.18 |
| 23 | 28.5 | 3.1 | 438 | 276 | 0.16 |
| 24 | 28.8 | 3.1 | 176 | 71 | 0.08 |
| 25 | 29.8 | 3.0 | 266 | 153 | 0.28 |
| 26 | 30.3 | 2.9 | 123 | 50 | 0.24 |
| 27 | 32.0 | 2.8 | 258 | 142 | 0.22 |
| 28 | 32.3 | 2.8 | 360 | 222 | 0.20 |
| 29 | 32.8 | 2.7 | 190 | 89 | 0.44 |
| 30 | 34.0 | 2.6 | 123 | 47 | 0.28 |
| 31 | 34.8 | 2.6 | 134 | 60 | 0.22 |
| 32 | 35.1 | 2.6 | 125 | 50 | 0.06 |
| 33 | 35.3 | 2.5 | 137 | 56 | 0.18 |
| 34 | 36.2 | 2.5 | 145 | 59 | 0.20 |
| 35 | 36.9 | 2.4 | 157 | 74 | 0.24 |

### Biological Data

### Materials:

H-Ala-Pro-pNA*HCl was purchased from BACHEM Bioscience Inc. (product no. L-1115). A 500 mM stock solution was prepared with dimethylsulfoxide and stored at −20° C. Gly-Pro-AMC was purchased from Enzyme System Products (product no. AMC-39) and stored at −20° C. as a 10 mM stock solution in dimethylsulfoxide. Test compound was dissolved to 10 mM in dimethylsulfoxide and this was used as a stock solution for DPP-IV titration assay. Athens Research and Technology, Inc prepared the purified human DPP-IV. The material was isolated from human prostasomes using the method of DeMeester et al., J. Immunol. Methods 189, 99-105. (1996), incorporated herein by reference to the extent of describing such method.

### DPP-IV Assay:

Two-fold serial dilutions of test compounds in 100% dimethylsulfoxide were performed in 96-well polystyrene flat bottom plates (Costar, #9017). The average enzymatic activity from wells containing dimethylsulfoxide but lacking test compound was used as a control value for calculating percent inhibition. DPP-IV (20 ng/mL) was mixed in microtiter plates with test compound, substrate and assay buffer to yield 100 μM H-Ala-Pro-pNA.HCl in 25 mM Tris, pH 7.5, 10 mM KCl, 140 mM NaCl. The intact peptide contains a p-nitrophenylanilide which, when hydrolyzed by DPP-IV, releases the absorbent p-nitrophenylaniline. The absorbency was monitored in 20 minutes intervals at a wavelength of 387 nm using a Molecular Devices SpectraMax 250 absorbency plate reader. The enzymatic activity was determined by estimating the best linear fit to the data. Values for enzymatic activity were taken directly from the linear fit determined by the software on the plate reader.

Data Analysis The enzymatic activity was determined by estimating the best linear fit to the data. Data reduction was performed using the Microsoft Excel RoboSage.

Determination of IC₅₀ values: The enzymatic activity was plotted against the concentration of test compound, including [I]=0, and the IC₅₀ determined from a fit of equation (2) to the data.

RATE=V_{max/(}1+([I]/IC₅₀)) (2)

Vₘₐₓ was the best fit estimate of the maximal enzymatic activity.

Determination of Kᵢ values: Kᵢ values were calculated from IC₅₀ values using equation (3) assuming a competitive model.

$${K}_{i} = I {C}_{50} * [ 1 - \frac{S}{( S + {K}_{m} )} ] \qquad ( 3 )$$

The apparent pKi values for the test compound was >5.0.

1-22. (canceled)

23. A compound comprising a crystalline form of a benzenesulfonic acid salt of trans-4-({2-[(2S)-2-cyanopyrrolidinyl]-2-oxoethyl}amino-N,N-dimethylcyclohexanecarboxamide.

24. The compound of claim 23, wherein said crystalline form of a benzene sulfonic acid salt is an anhydrate.

25. The compound of claim 24, wherein said crystalline form of a benzenesulfonic acid salt exhibits a melting point of 157° C.

26. The compound of claim 24, wherein said crystalline form of a benzenesulfonic acid salt is obtainable by the reaction of trans-4-({2-[(2S)-2-cyanopyrrolidinyl]-2oxoethyl}-amino)-N,N-dimethylcyclohexanecarboxamide with benzene sulfonic acid and crystallization in a polar solvent.

27. A compound, comprising a crystalline form of an anhydrous besylate salt of trans-4-({2-[(2S)-2-cyanopyrrolidinyl]-2-oxoethyl}amino)-N,N-dimethylcyclohexanecarboxamide, wherein said crystalline form exhibits a powder x-ray diffraction pattern comprising the following peaks:

| Two theta (deg) | d-spacing (angstroms) |
|---|---|
| 6.1 ± 0.2 | 14.5 ± 0.5 |
| 14.1 ± 0.2 | 6.3 ± 0.1 |
| 16.3 ± 0.2 | 5.4 ± 0.1 |
| 19.8 ± 0.2 | 4.5 ± 0.1 |
| 22.4 ± 0.2 | 4.0 ± 0.1 |

28. The compound of claim 23, wherein said crystalline form of a benzenesulfonic acid salt is a monohydrate.

29. The compound of claim 28, wherein said crystalline form of a benzenesulfonic acid salt exhibits a melting point ranging from 110 to 120° C.

30. The compound of claim 28, wherein said crystalline form of a benzene sulfonic acid salt is obtainable by the reaction of trans-4-({2-[(2S)-2-cyanopyrrolidinyl]-2-oxoethyl}amino)-N,N-dimethylcyclohexanecarboxamide with benzene sulfonic acid and crystallization in a mixture of a polar solvent and water.

31. A compound comprising a crystalline form of a monohydrate besylate salt of the compound, trans-4-({2-[(2S)-2-cyanopyrrolidinyl]-2-oxoethyl}amino)-N,N-dimethylcyclohexanecarboxamide, wherein said crystalline form exhibits a powder x-ray diffraction pattern comprising the following peaks:

| Two theta (deg) | d-spacing (angstroms) |
|---|---|
| 6.8 ± 0.2 | 13.0 ± 0.4 |
| 7.8 ± 0.2 | 11.3 ± 0.3 |
| 8.8 ± 0.2 | 10.0 ± 0.3 |
| 17.4 ± 0.2 | 5.1 ± 0.1 |
| 26.7 ± 0.2 | 3.3 ± 0.1 |

32. A process for preparing the compound of claim 23 or 24, comprising forming said crystalline form of a benzenesulfonic acid salt by reacting trans-4-({2-[(2S)-2-cyanopyrrolidinyl]-2-oxoethyl}amino)-N,N-dimethylcyclohexanecarboxamide with benzenesulfonic acid, followed by crystallization in a polar solvent.

33. A process for preparing the compound of claims 23 or 28, comprising forming said benzenesulfonic acid salt by reaction of trans-4-({2-[(2S)-2-cyanopyrrolidinyl]-2-oxoethyl}amino)-N,N-dimethylcyclohexanecarboxamide with benzenesulfonic acid, followed by crystallization in a mixture of a polar solvent and water.

34. A pharmaceutical composition, comprising:
a therapeutically effective amount of a compound according to any one of claims 23-31 or 39, and
one or more of pharmaceutically acceptable carriers, diluents and excipients.

35. A pharmaceutical composition comprising:
a crystalline form of trans-4-({2-[(2S)-2-cyanopyrrolidinyl]-2-oxoethyl}amino)-N,N-dimethylcyclohexanecarboxamide besylate anhydrate according to claim 27; or
a crystalline of trans-4-({2-[(2S)-2-cyanopyrrolidinyl]-2-oxoethyl}amino)-N,N-dimethylcyclohexanecarboxamide besylate monohydrate according to claim 31; or
a mixture thereof.

36. The pharmaceutical composition of claim 34, comprising at least one hydrate of trans-4-({2-[(2S)-2-cyanopyrrolidinyl]-2-oxoethyl}amino)-N,N-dimethylcyclohexanecarboxamide besylate.

37. The pharmaceutical composition of claim 34, further comprising at least one solvate of trans-4-({2-[(2S)-2-cyanopyrrolidinyl]-2-oxoethyl}amino)-N,N-dimethylcyclohexanecarboxamide besylate.

38. The compound of any one of claims 23-31 or 39, for use in medical therapy.

39. The compound of claim 23, wherein said crystalline form of a benzenesulfonic acid salt is a crystalline anhydrate, hydrate, or solvate.
